(19) United States
(12) Patent Application Publication
(10) Pub. No.: US 2025/0375397 A1
(43) Pub. Date: Dec. 11, 2025

(54) Topical Patch Device

(71) Applicant: James Bain, Ingleside, IL (US)

(72) Inventor: James Bain, Ingleside, IL (US)

(21) Appl. No.: 19/022,104

(22) Filed: Jan. 15, 2025

Related U.S. Application Data
(60) Provisional application No. 63/656,628, filed on Jun. 6, 2024.

(51) Int. Cl. A61K 9/70

(52) U.S. Cl. CPC A61K 9/7084

(57) ABSTRACT

A topical patch device is disclosed, which is designed for the relief and control of psoriasis through delivering medication directly into the skin. The topical patch device comprises a body component which is a patch that is configured in a variety of shapes. The body component comprises medicinal ingredients designed to treat psoriasis. Further, the body component comprises a protective adhesive backing that can be peeled away once application is needed to expose the medicinal ingredients. Users can then secure the patch to an affected area to obtain a better, more consistent method of absorbing medication when compared to foams and lotions.

### CROSS-REFERENCE TO RELATED APPLICATION

The present application claims priority to, and the benefit of, U.S. Provisional Application No. 63/656,628, which was filed on Jun. 6, 2024, and is incorporated herein by reference in its entirety.

### FIELD OF THE INVENTION

The present invention relates generally to the field of topical patch devices. More specifically, the present invention relates to a topical patch infused with medication that helps treat plaque psoriasis. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in topical patch devices. Generally, plaque psoriasis is a chronic autoimmune condition that affects the skin, causing red, raised, scaly patches known as plaques. These plaques can appear anywhere on the body but are commonly found on the elbows, knees, scalp, and lower back. Classical symptoms cycle through periods of activity and remission, and the implications of active psoriasis can range from minor itching to life-threatening immune system overload. The visibility of the outbreaks and the ability of the disease to become resistant to previously effective treatments can cause psychological repercussions as well.

Further, treatment for plaque psoriasis can vary depending on the severity of the condition and individual factors and include topical treatments, phototherapy, systemic medications, and even alternative therapies. The background of the art reveals a significant deficiency in topical treatments with various agents because the modes of delivery have proven unreliable. Further, while effective, topical treatments can be messy and inconvenient. However, lack of treatment leads to severe discomfort, itching, redness, and decreased self-esteem due to the scaly look of the skin.

Accordingly, there is a demand for an improved topical patch device that provides users with a topical patch infused with medication to help treat plaque psoriasis. More particularly, there is a demand for a topical patch device that reduces the mess associated with treating psoriasis.

Therefore, there exists a long felt need in the art for a topical patch device that provides users with a topical patch infused with medication that helps treat plaque psoriasis. There is also a long felt need in the art for a topical patch device that utilizes a combination of specific ingredients to treat redness, itchiness, and discomfort associated with psoriasis. Further, there is a long felt need in the art for a topical patch device that offers quicker and more effective relief compared to foam or lotions that can take some time to treat the symptoms. Moreover, there is a long felt need in the art for a device that reduces the mess associated with treating psoriasis compared to standard foams, lotions, and ointments. Further, there is a long felt need in the art for a topical patch device that can be secured to the affected area. Finally, there is a long felt need in the art for a topical patch device that includes a protective adhesive backing.

The subject matter disclosed and claimed herein, in one embodiment thereof, comprises a topical patch device. The device is a topical patch designed for the relief and control of psoriasis through delivering medication directly into the skin. The topical patch device comprises a body component which is a patch that is configured in a variety of shapes. The body component comprises medicinal ingredients designed to treat psoriasis. Further, the body component comprises a protective adhesive backing that can be peeled away once application is needed to expose the medicinal ingredients. Users can then secure the patch to an affected area to obtain a better, more consistent method of absorbing medication when compared to foams and lotions.

In this manner, the topical patch device of the present invention accomplishes all of the forgoing objectives and provides users with a topical patch that delivers medication to treat psoriasis. The device includes medicinal ingredients for treating psoriasis. The device is applied directly to the user's skin.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a topical patch device. The device is a topical patch designed for the relief and control of psoriasis through delivering medication directly into the skin. The topical patch device comprises a body component which is a patch that is configured in a variety of shapes. The body component comprises medicinal ingredients designed to treat psoriasis. Further, the body component comprises a protective adhesive backing (i.e., release liner) that can be peeled away once application is needed to expose the medicinal ingredients and an adhesive area for adhering to the skin. Users can then secure the patch to an affected area to obtain a better, more consistent method of absorbing medication when compared to foams and lotions.

In one embodiment, the topical patch device utilizes a combination of specific ingredients to treat redness, itchiness, and discomfort associated with psoriasis. Further, the use of the topical patch device reduces the mess associated with treating psoriasis compared to standard foams, lotions, and ointments. Accordingly, the topical patch device offers quicker and more effective relief compared to foam or lotions that can take some time to treat the symptoms. Further, because psoriasis and other skin disorders manifests as itchy, flaky skin plaques known to innervate all different epithelium strata, the present invention contemplates a need to treat all layers of the epithelium strata.

In one embodiment, the topical patch device comprises a body component which is configured as a patch to be applied over an affected area on a user's skin. The body component is applied to an affected area of a user's skin by anyone including the user themselves and which contains medicinal ingredients carried by an absorbent material, such as cotton, and which contains both active and inactive ingredients for the purpose of applying the medicinal ingredients to treat plaque psoriasis. Typically, the body component can be any suitable shape and size depending on the size and shape of the affected area to be treated. Further, while shown as rectangular in the drawings, may be of any other convenient geometric shape as is known in the art. In addition, the body component can be cut, as desired, to provide a patch of any suitable size and shape. The body component can be cut with any suitable cutting device such as a scissors, scalpel, or knife, etc.

In one embodiment, the body component is typically a flexible porous sheet of water insoluble material that provides support for a gauze pad (described below). The body component should be nonirritating to human skin. The body component can be vapor permeable. Specifically, the body component can retain the medicinal ingredients while allowing moisture from the skin to pass. The body component can be woven or nonwoven. Preferably, the body component can include polyester, polyurethane, polyolefin, polyamide fibers, natural fibers, cotton fibers, poly cellulose fibers, or any mixture thereof. The body component can have any suitable thickness, provided the suitable thickness allows for a flexible, bendable, pliable, vapor permeable, and/or a stretchable sheet of water insoluble porous material. Specifically, the thickness of the body component can be about 0.001 mm to about 3.0 mm, or about 0.025 mm to about 1.25 mm.

In one embodiment, the patch device, upon contact with skin, will allow the skin to breathe. More preferably, the patch device, upon prolonged contact with skin, will hold in place the medicinal ingredients and will permit the skin to breathe over prolonged periods of time typically experienced with the use of the patch device, i.e., up to about 12 hours, up to about 8 hours, or up to about 6 hours, etc.

In one embodiment, the present invention provides an ointment or gel on the body component. The ointment or gel includes an effective, known, and safe amount of a medicament that is useful for treating psoriasis and a pressure sensitive adhesive to secure it to a user's skin. Further, the body component is pliable and/or stretchable. Since the body component can be porous and/or vapor permeable, many consumers typically refer to the device as a “patch.” As such, the device will herein be referred to as a patch, as a skin patch, and/or as a topical patch. It is appreciated that those skilled in the art understand that the term patch is used to refer to the device and is not otherwise limiting in any manner.

In one embodiment, the topical patch device administers to the skin an effective and known amount of a topical psoriasis medication(s). The patch device maintains the adhesiveness of the adhesive and the stability of the topical psoriasis medication(s) over a prolonged period of time typically experienced in the manufacturing, packaging, shipping, and/or the storage of the patch device. The topical psoriasis medication(s) and pressure sensitive adhesive are positioned on and in at least a portion of the patch device, such that they are partially embedded in at least a portion of the patch device.

In one embodiment, the body component comprises a front surface which is applied to the affected area and a rear surface which faces away from the affected area. In another embodiment, the body component includes a gauze pad, attached to the front surface, wherein the gauze pad can have an impermeable side which contacts the front surface, and an absorbent top side which contacts the user's skin.

In one embodiment, the gauze pad of the front surface of the body component comprises medicinal ingredients designed to treat psoriasis. Specifically, the body component allows for the application of the medicinal ingredients to the afflicted psoriatic skin plaque and the immediate surrounding skin region. The medicinal ingredients can be any suitable medicinal ingredients, including active and inactive ingredients, as is known in the art which are configured for direct topical application to the most surficial epidermal layer of a user's skin. Further, it should be understood that the various concentrations of the active or inactive ingredients can be varied to alter duration or intensity or other affectable properties. In one embodiment, analgesic agents and drying agents can be included on the body component. Specifically, the analgesic agent is selected to reduce inflammation, pain, and irritation surrounding the superficial skin plaques, which can lead to further irritation and subsequent outbreaks. The analgesic agents also help to inhibit the cyclo-oxygenase enzyme, which is important in order to minimize pain and inflammation. Typically, the analgesic agent is selected from the group of salicylic acid and ibuprofen, which share the additional common characteristics of anti-inflammation, rapid effect, and relatively short half-life. These agents also have an established record on bioavailability and low toxicity.

In one embodiment, the drying agent is necessary to help modulate the rampant growth of the stratum basale and to minimize water encapsulated within the stratum spinosum and the stratum granulosum, which in turn fosters a detachment of the psoriatic plaques from, for example, the stratum lucidum and the stratum corneum, i.e., the upper skin strata. Psoriatic plaques innervate not simply the stratum corneum and the stratum ludicum but have been shown to incorporate deeper skin layers as the origination of psoriasis is known to begin with the stratum basale. The drying agent also serves a second purpose, which is to assist in the delivery of the analgesic agent to the deepest layers of the skin epidermis. One or more drying agents are typically selectively chosen from the group of ethyl alcohol, isopropyl alcohol, ethyl benzene, and m-xylene, o-xylene, and p-xylene. The present invention can incorporate one or more of these listed agents to dehydrate and exfoliate psoriatic plaques to induce the growth of normal, healthy skin. Typically, the drying agents share common characteristics originating with chemical structure and polarity and have similar effects upon lipid-based membranes that form the cellular structure of the skin.

In one embodiment, the front surface of the body component which contacts the user's skin, also comprises at least one section of a pressure sensitive adhesive. Any suitable amount of pressure sensitive adhesive can be utilized as needed, depending on the shape and size of the body component. For example, the pressure sensitive adhesive can be located on the entire surface of the front surface or the pressure sensitive adhesive can be located on a portion of the front surface. When the patch device is placed upon the skin of a user, the pressure sensitive adhesive is in continuous contact with the user's skin surface. Further, any suitable pressure sensitive adhesive can be employed, provided the pressure sensitive adhesive provides the requisite adhesiveness to the patch device and the pressure sensitive adhesive remains stable in the medicinal ingredients. Preferably, the stability is over a prolonged period of time, i.e., up to about 3 years, up to about 1 year, or up to about 6 months, typically experienced in the manufacturing, packaging, shipping, and/or storage of the patch device.

In one embodiment, the topical patch device can be attached to a release liner. The release liner helps to maintain the adhesiveness of the patch device prior to use, such as during manufacturing, packaging, shipping, and/or storage. The release liner is applied over the medicinal ingredients on the gauze pad and any adhesive area and is removed prior to application on a user. Any suitable release liner can be employed for use in the present invention.

In one embodiment, the entire patch device described above is preferably covered with a plastic or foil wrapping to retain moisture and retard evaporation. Further, the entire patch device is packaged as a sterile, or as other over-the-counter adhesive bandages.

In one embodiment, the topical patch device is applied to the skin surface of a user. The topical patch device can be applied to any suitable skin surface of the user. Suitable skin surfaces in which the patch device can be applied include, i.e., the face, neck, shoulder, chest, and back.

In one embodiment, the topical patch device serves as a protective covering or barrier. Such protection serves to prevent or diminish the likelihood that foreign objects (i.e., a person's finger, hair, clothing, etc.) will come into contact with the affected psoriasis area. This may effectively decrease the healing time of the affected psoriasis area.

In one embodiment, the topical patch device also serves to aesthetically cover psoriasis areas of the skin, as many individuals are self-conscious and attempt to cover the affected psoriasis area. The topical patch device allows users to completely cover the affected psoriasis area, thereby concealing the affected area, while effectively treating the psoriasis. As such, the topical patch device can serve to aesthetically cover psoriasis areas of the skin for prolonged periods of time (i.e., up to about 8 hours, up to about 6 hours, or up to about 4 hours, etc.).

In operation, the topical patch device is removed from its protective device, such as the plastic or foil wrapping, and is placed on a user's skin, at a predetermined time, and most likely a predetermined site, with the absorbent gauze pad contacting the skin. At this time, the skin area under the gauze pad will come into contact with the medicinal ingredients. When the patch device is removed from the skin, it is disposed of.

In yet another embodiment, the topical patch device comprises a plurality of indicia.

In yet another embodiment, a method of delivering psoriasis medication to a user via a patch is disclosed. The method includes the steps of providing a topical patch device comprising a body component configured as a patch with a protective adhesive backing secured over a medicinal area and an adhesive border. The method also comprises choosing the right size and shape of the topical patch device as needed to treat the plaque psoriasis. Further, the method comprises removing the protective adhesive backing. The method also comprises positioning the exposed medicinal side of the patch onto the affected area of a user's skin. Finally, the method comprises securing the topical patch device to the affected area via pressing on the back of the body component to firmly adhere the adhesive border around the affected area.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the topical patch device of the present invention showing the device in use in accordance with the disclosed architecture;

FIG. 2 illustrates a perspective view of one embodiment of the topical patch device of the present invention showing the patch being peeled away from the protective adhesive backing in accordance with the disclosed architecture;

FIG. 3 illustrates a perspective view of one embodiment of the topical patch device of the present invention showing untreated plaque psoriasis on the back of a hand in accordance with the disclosed architecture;

FIG. 4 illustrates a perspective view of one embodiment of the topical patch device of the present invention showing how the patch is applied in accordance with the disclosed architecture;

FIG. 5 illustrates a perspective view of one embodiment of the topical patch device of the present invention showing the patch releasing medicine in accordance with the disclosed architecture; and

FIG. 6 illustrates a flowchart showing the method of delivering psoriasis medication to a user via a patch in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a topical patch device that provides users with a topical patch infused with medication that helps treat plaque psoriasis. There is also a long felt need in the art for a topical patch device that utilizes a combination of specific ingredients to treat redness, itchiness, and discomfort associated with psoriasis. Further, there is a long felt need in the art for a topical patch device that offers quicker and more effective relief compared to foam or lotions that can take some time to treat the symptoms. Moreover, there is a long felt need in the art for a device that reduces the mess associated with treating psoriasis compared to standard foams, lotions, and ointments. Further, there is a long felt need in the art for a topical patch device that can be secured to the affected area. Finally, there is a long felt need in the art for a topical patch device that includes a protective adhesive backing.

The present invention, in one exemplary embodiment, is a novel topical patch device. The device is a topical patch designed for the relief and control of psoriasis through delivering medication directly into the skin. The topical patch device comprises a body component which is a patch that is configured in a variety of shapes. The body component comprises medicinal ingredients designed to treat psoriasis. Further, the body component comprises a protective adhesive backing that can be peeled away once application is needed to expose the medicinal ingredients. Users can then secure the patch to an affected area via an adhesive border, to obtain a better, more consistent method of absorbing medication when compared to foams and lotions. The present invention also includes a novel method of delivering psoriasis medication to a user via a patch. The method includes the steps of providing a topical patch device comprising a body component configured as a patch with a protective adhesive backing secured over a medicinal area and an adhesive border. The method also comprises choosing the right size and shape of the topical patch device as needed to treat the plaque psoriasis. Further, the method comprises removing the protective adhesive backing. The method also comprises positioning the exposed medicinal side of the patch onto the affected area of a user's skin. Finally, the method comprises securing the topical patch device to the affected area via pressing on the back of the body component to firmly adhere the adhesive border around the affected area.

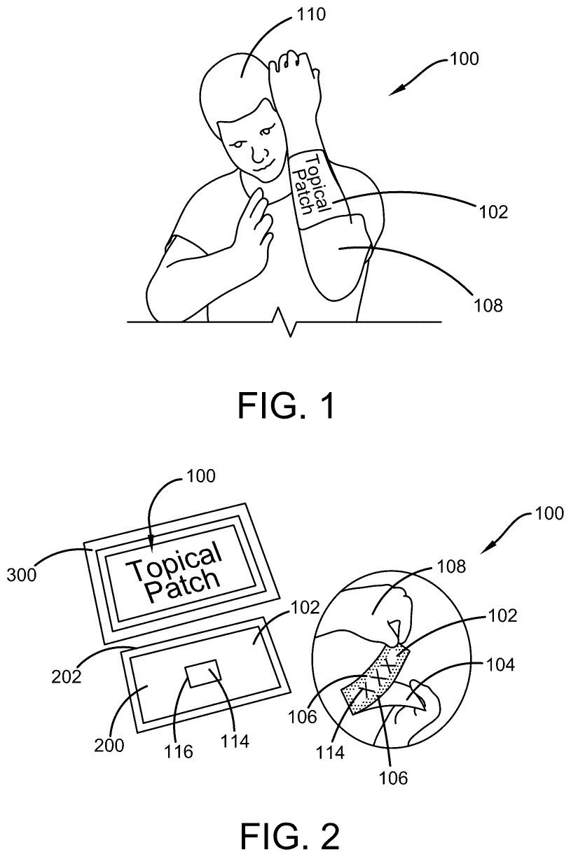

Referring initially to the drawings, FIG. 1 illustrates a perspective view of one embodiment of the topical patch device 100 of the present invention. In the present embodiment, the topical patch device 100 is an improved topical patch device 100 that provides a user with a topical patch for delivering medicinal ingredients designed to treat psoriasis. Specifically, the topical patch device 100 comprises a body component 102, an attached protective adhesive backing 104 (i.e., release liner) and an adhesive area 106 for adhering to the skin 108. Users 110 can then secure the patch device 100 to an affected area to obtain a better, more consistent method of absorbing medication when compared to foams and lotions.

Generally, the topical patch device 100 utilizes a combination of specific ingredients to treat redness, itchiness, and discomfort associated with psoriasis. Further, the use of the topical patch device 100 reduces the mess associated with treating psoriasis compared to standard foams, lotions, and ointments. Accordingly, the topical patch device 100 offers quicker and more effective relief compared to foam or lotions that can take some time to treat the symptoms. Further, because psoriasis and other skin disorders manifests as itchy, flaky skin plaques known to innervate all different epithelium strata, the present invention contemplates a need to treat all layers of the epithelium strata.

Further, the topical patch device 100 comprises a body component 102 which is configured as a patch to be applied over an affected area 112 on a user's skin 108. The body component 102 is applied to an affected area 112 of a user's skin 108 by anyone including the user 110 themselves and which contains medicinal ingredients 114 carried by an absorbent material, such as cotton, and which contains both active and inactive ingredients for the purpose of applying the medicinal ingredients 114 to treat plaque psoriasis. Typically, the body component 102 can be any suitable shape and size depending on the size and shape of the affected area to be treated. Further, while shown as rectangular in the drawings, may be of any other convenient geometric shape as is known in the art. In addition, the body component 102 can be cut, as desired, to provide a patch of any suitable size and shape. The body component 102 can be cut with any suitable cutting device such as a scissors, scalpel, or knife, etc.

Additionally, the body component 102 is typically a flexible porous sheet of water insoluble material that provides support for a gauze pad 116 (described below). The body component 102 should be nonirritating to human skin 108. The body component 102 can be vapor permeable. Specifically, the body component 102 can retain the medicinal ingredients 114 while allowing moisture from the skin 108 to pass. The body component 102 can be woven or nonwoven. Preferably, the body component 102 can include polyester, polyurethane, polyolefin, polyamide fibers, natural fibers, cotton fibers, poly cellulose fibers, or any mixture thereof. The body component 102 can have any suitable thickness, provided the suitable thickness allows for a flexible, bendable, pliable, vapor permeable, and/or a stretchable sheet of water insoluble porous material. Specifically, the thickness of the body component 102 can be about 0.001 mm to about 3.0 mm, or about 0.025 mm to about 1.25 mm.

Furthermore, the patch device 100, upon contact with skin 108, will allow the skin 108 to breathe. More preferably, the patch device 100, upon prolonged contact with skin 108, will hold in place the medicinal ingredients 114 and will permit the skin 108 to breathe over prolonged periods of time typically experienced with the use of the patch device 100, i.e., up to about 12 hours, up to about 8 hours, or up to about 6 hours, etc.

As shown in FIG. 2, the present invention provides an ointment or gel on the body component 102. The ointment or gel includes an effective, known, and safe amount of a medicament 114 that is useful for treating psoriasis and a pressure sensitive adhesive 106 to secure it to a user's skin 108. Further, the body component 102 is pliable and/or stretchable. Since the body component 102 can be porous and/or vapor permeable, many consumers typically refer to the device 100 as a “patch.” As such, the device 100 will herein be referred to as a patch, as a skin patch, and/or as a topical patch. It is appreciated that those skilled in the art understand that the term patch is used to refer to the device 100 and is not otherwise limiting in any manner.

Further, the topical patch device 100 administers to the skin 108 an effective and known amount of a topical psoriasis medication(s) 114. The patch device 100 maintains the adhesiveness of the adhesive 106 and the stability of the topical psoriasis medication(s) 114 over a prolonged period of time typically experienced in the manufacturing, packaging, shipping, and/or the storage of the patch device 100. The topical psoriasis medication(s) 114 and pressure sensitive adhesive 106 are positioned on and in at least a portion of the patch device 100, such that they are partially embedded in at least a portion of the patch device 100.

Additionally, the body component 102 comprises a front surface 200 which is applied to the affected area 112 and a rear surface 202 which faces away from the affected area 112. In another embodiment, the body component 102 includes a gauze pad 116, attached to the front surface 200, wherein the gauze pad 116 can have an impermeable side which contacts the front surface 200, and an absorbent top side which contacts the user's skin 108.

Furthermore, the gauze pad 116 of the front surface 200 of the body component 102 comprises medicinal ingredients 114 designed to treat psoriasis. Specifically, the body component 102 allows for the application of the medicinal ingredients 114 to the afflicted psoriatic skin plaque and the immediate surrounding skin region. The medicinal ingredients 114 can be any suitable medicinal ingredients, including active and inactive ingredients, as is known in the art which are configured for direct topical application to the most surficial epidermal layer of a user's skin 108. Further, it should be understood that the various concentrations of the active or inactive ingredients can be varied to alter duration or intensity or other affectable properties. In one embodiment, analgesic agents and drying agents can be included on the body component 102. Specifically, the analgesic agent is selected to reduce inflammation, pain, and irritation surrounding the superficial skin plaques, which can lead to further irritation and subsequent outbreaks. The analgesic agents also help to inhibit the cyclo-oxygenase enzyme, which is important in order to minimize pain and inflammation. Typically, the analgesic agent is selected from the group of salicylic acid and ibuprofen, which share the additional common characteristics of anti-inflammation, rapid effect, and relatively short half-life. These agents also have an established record on bioavailability and low toxicity.

In one embodiment, the drying agent is necessary to help modulate the rampant growth of the stratum basale and to minimize water encapsulated within the stratum spinosum and the stratum granulosum, which in turn fosters a detachment of the psoriatic plaques from, for example, the stratum lucidum and the stratum corneum, i.e., the upper skin strata. Psoriatic plaques innervate not simply the stratum corneum and the stratum ludicum but have been shown to incorporate deeper skin layers as the origination of psoriasis is known to begin with the stratum basale. The drying agent also serves a second purpose, which is to assist in the delivery of the analgesic agent to the deepest layers of the skin epidermis. One or more drying agents are typically selectively chosen from the group of ethyl alcohol, isopropyl alcohol, ethyl benzene, and m-xylene, o-xylene, and p-xylene. The present invention can incorporate one or more of these listed agents to dehydrate and exfoliate psoriatic plaques to induce the growth of normal, healthy skin. Typically, the drying agents share common characteristics originating with chemical structure and polarity and have similar effects upon lipid-based membranes that form the cellular structure of the skin.

Furthermore, the front surface 200 of the body component 102 which contacts the user's skin 108, also comprises at least one section of a pressure sensitive adhesive 106. Any suitable amount of pressure sensitive adhesive 106 can be utilized as needed, depending on the shape and size of the body component 102. For example, the pressure sensitive adhesive 106 can be located on the entire surface of the front surface 200 or the pressure sensitive adhesive 106 can be located on a portion of the front surface 200. When the patch device 100 is placed upon the skin 108 of a user 110, the pressure sensitive adhesive 106 is in continuous contact with the user's skin surface 108. Further, any suitable pressure sensitive adhesive 106 can be employed, provided the pressure sensitive adhesive 106 provides the requisite adhesiveness to the patch device 100 and the pressure sensitive adhesive 106 remains stable in the medicinal ingredients 114. Preferably, the stability is over a prolonged period of time, i.e., up to about 3 years, up to about 1 year, or up to about 6 months, typically experienced in the manufacturing, packaging, shipping, and/or storage of the patch device 100.

Additionally, the topical patch device 100 can be attached to a release liner 104. The release liner 104 helps to maintain the adhesiveness of the patch device 100 prior to use, such as during manufacturing, packaging, shipping, and/or storage. The release liner 104 is applied over the medicinal ingredients 114 on the gauze pad 116 and any adhesive area 106 and is removed prior to application on a user 110. Any suitable release liner 104 can be employed for use in the present invention.

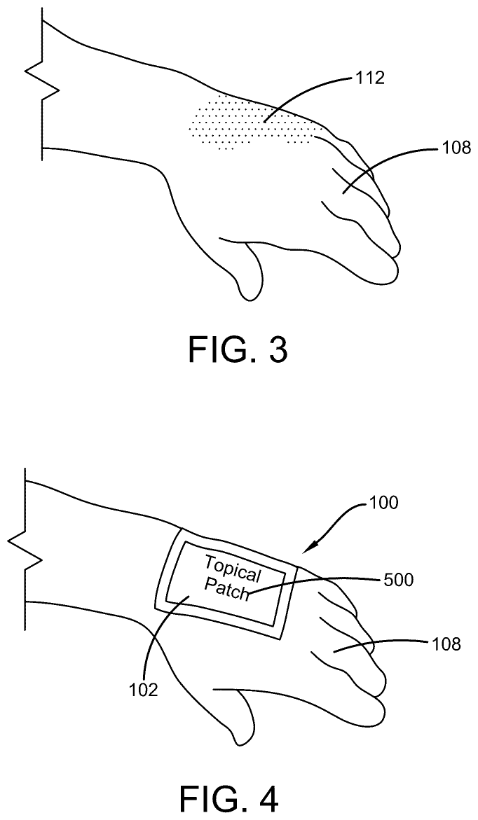

As shown in FIGS. 3-4, the entire patch device 100 described above is preferably covered with a plastic or foil wrapping 300 to retain moisture and retard evaporation. Further, the entire patch device 100 is packaged as a sterile, or as other over-the-counter adhesive bandages.

Further, the topical patch device 100 is applied to the skin 108 surface of a user 110. The topical patch device 100 can be applied to any suitable skin surface 108 of the user 110. Suitable skin surfaces 108 in which the patch device 100 can be applied include, i.e., the face, neck, shoulder, chest, and back.

In one embodiment, the topical patch device 100 serves as a protective covering or barrier. Such protection serves to prevent or diminish the likelihood that foreign objects (i.e., a person's finger, hair, clothing, etc.) will come into contact with the affected psoriasis area 112. This may effectively decrease the healing time of the affected psoriasis area 112.

In one embodiment, the topical patch device 100 also serves to aesthetically cover psoriasis areas 112 of the skin 108, as many individuals are self-conscious and attempt to cover the affected psoriasis area 112. The topical patch device 100 allows users 110 to completely cover the affected psoriasis area 112, thereby concealing the affected area 112, while effectively treating the psoriasis. As such, the topical patch device 100 can serve to aesthetically cover psoriasis areas 112 of the skin 108 for prolonged periods of time (i.e., up to about 8 hours, up to about 6 hours, or up to about 4 hours, etc.).

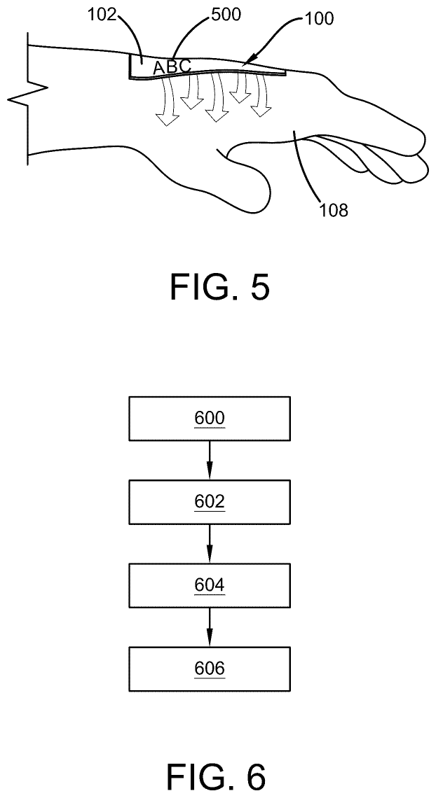

As shown in FIG. 5, in operation, the topical patch device 100 is removed from its protective device, such as the plastic or foil wrapping 300, and is placed on a user's skin 108, at a predetermined time, and most likely a predetermined site, with the absorbent gauze pad 116 contacting the skin 108. At this time, the affected area 112 under the gauze pad 116 will come into contact with the medicinal ingredients 114. When the patch device 100 is removed from the skin 108, it is disposed of.

In yet another embodiment, the topical patch device 100 comprises a plurality of indicia 500. The body component 102 of the device 100 may include advertising, a trademark, or other letters, designs, or characters, printed, painted, stamped, or integrated into the body component 102, or any other indicia 500 as is known in the art. Specifically, any suitable indicia 500 as is known in the art can be included, such as but not limited to, patterns, logos, emblems, images, symbols, designs, letters, words, characters, animals, advertisements, brands, etc., that may or may not be patch, psoriasis, or brand related.

FIG. 6 illustrates a flowchart of the method of delivering psoriasis medication to a user via a patch. The method includes the steps of at 600, providing a topical patch device comprising a body component configured as a patch with a protective adhesive backing secured over a medicinal area and an adhesive border. The method also comprises at 602, choosing the right size and shape of the topical patch device as needed to treat the plaque psoriasis. Further, the method comprises at 604, removing the protective adhesive backing. The method also comprises at 606, positioning the exposed medicinal side of the patch onto the affected area of a user's skin. Finally, the method comprises at 606, securing the topical patch device to the affected area via pressing on the back of the body component to firmly adhere the adhesive border around the affected area.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “topical patch device”, “patch device”, and “device” are interchangeable and refer to the topical patch device 100 of the present invention.

Notwithstanding the forgoing, the topical patch device 100 of the present invention can be of any suitable size and configuration as is known in the art without affecting the overall concept of the invention, provided that it accomplishes the above stated objectives. One of ordinary skill in the art will appreciate that the topical patch device 100 as shown in FIGS. 1-6 is for illustrative purposes only, and that many other sizes and shapes of the topical patch device 100 are well within the scope of the present disclosure. Although the dimensions of the topical patch device 100 are important design parameters for user convenience, the topical patch device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A topical patch device that provides a user with a topical patch for delivering medicinal ingredients, the topical patch device comprising:
a body component;
a release liner;
an adhesive area; and
medicinal ingredients;
wherein the medicinal ingredients are utilized to treat psoriasis;
wherein the medicinal ingredients are embedded within the body component;
wherein the adhesive area is disposed on the body component;
wherein the release liner covers and protects the medicinal ingredients and the adhesive area and is removed prior to use; and
further wherein the body component is applied on a user's skin and the medicinal ingredients are positioned over an affected psoriasis area to treat the affected psoriasis area.

2. The topical patch device of claim 1, wherein the body component is configured as a patch to be applied over the affected psoriasis area on a user's skin.

3. The topical patch device of claim 2, wherein the medicinal ingredients contain both active and inactive ingredients for treating plaque psoriasis.

4. The topical patch device of claim 3, wherein the body component can be any suitable shape and size and can be cut, as desired.

5. The topical patch device of claim 4, wherein the body component is a flexible porous sheet of water insoluble material that provides support for a gauze pad.

6. The topical patch device of claim 5, wherein the body component comprises a front surface which is applied to the affected psoriasis area and a rear surface which faces away from the affected psoriasis area.

7. The topical patch device of claim 6, wherein the gauze pad is attached to the front surface of the body component and comprises the medicinal ingredients designed to treat psoriasis.

8. The topical patch device of claim 7, wherein the adhesive area includes a pressure sensitive adhesive.

9. The topical patch device of claim 8, wherein the pressure sensitive adhesive can be located on an entire surface of the front surface or the pressure sensitive adhesive can be located on a portion of the front surface.

10. The topical patch device of claim 9, wherein the release liner helps to maintain adhesiveness of the topical patch device prior to use, such as during manufacturing, packaging, shipping, and storage, and is applied over the medicinal ingredients on the gauze pad and any adhesive area and is removed prior to application on a user.

11. The topical patch device of claim 10, wherein an entire topical patch device is packaged in a foil wrapping.

12. A topical patch device that provides a user with a topical patch for delivering medicinal ingredients, the topical patch device comprising:
a body component comprising a front surface which is applied to an affected psoriasis area and a rear surface which faces away from the affected psoriasis area;
an adhesive area located on an entire surface of the front surface or located on a portion of the front surface;
medicinal ingredients containing both active and inactive ingredients for treating plaque psoriasis; and
a release liner to maintain adhesiveness of the topical patch device prior to use, such as during manufacturing, packaging, shipping, and storage, and is applied over the medicinal ingredients and any adhesive area and is removed prior to application on a user;
wherein the medicinal ingredients are embedded within the body component; and
further wherein the body component is applied on a user's skin and the medicinal ingredients are positioned over an affected psoriasis area to treat the affected psoriasis area.

13. The topical patch device of claim 12, wherein the body component is a flexible porous sheet of water insoluble material that provides support for a gauze pad that is attached to the front surface and comprises the medicinal ingredients.

14. The topical patch device of claim 12, wherein the body component can be any suitable shape and size and can be cut, as desired.

15. The topical patch device of claim 12, wherein an entire topical patch device is packaged in a foil wrapping.

16. The topical patch device of claim 12, wherein the topical patch device is applied to any suitable skin surface of a user, such as face, neck, shoulder, chest, and back.

17. The topical patch device of claim 12, wherein the topical patch device serves as a protective covering or barrier against foreign objects.

18. The topical patch device of claim 12, wherein the topical patch device serves to aesthetically cover an affected psoriasis area of a user's skin for prolonged periods of time.

19. The topical patch device of claim 12 further comprising a plurality of indicia.

20. A method of delivering psoriasis medication to a user via a patch, the method comprising the following steps:
providing a topical patch device comprising a body component configured as a patch with a protective adhesive backing secured over a medicinal area and an adhesive border;
choosing the right size and shape of the topical patch device as needed to treat the plaque psoriasis;
removing the protective adhesive backing;
positioning the exposed medicinal side of the patch onto the affected area of a user's skin; and
securing the topical patch device to the affected area via pressing on the back of the body component to firmly adhere the adhesive border around the affected area.
